(19) United States
(12) Patent Application Publication
(10) Pub. No.: US 2017/0184535 A1
(43) Pub. Date: Jun. 29, 2017

(54) CONDUCTING POLYMERS FOR DIRECT SENSING OF METAL IONS

(71) Applicant: BOARD OF REGENTS, THE UNIVERSITY OF TEXAS SYSTEM, Austin, TX (US)

(72) Inventor: Bradley J. Holliday, Austin, TX (US)

(21) Appl. No.: 15/124,493

(22) PCT Filed: Mar. 17, 2015
(86) PCT No.: PCT/US15/20924

§ 371 Date: Sep. 8, 2016

Related U.S. Application Data
(60) Provisional application No. 61/954,370, filed on Mar. 17, 2014.

(51) Int. Cl. G01N 27/333; C07D 409/14; H01B 1/20

(52) U.S. Cl. CPC G01N 27/3335; H01B 1/20; C07D 409/14

(57) ABSTRACT

The present invention provides an ion selective electrode comprising an electrode having a coating deposited on the electrode, wherein the coating comprises one or more aroyl-thiourea ionophores incorporated into a polymer matrix to selectively interact with one or more ions. The aroylthiourea ionophores may be poly-5, poly-6, poly-7, poly-7a, poly-7b, poly-8a, poly-8b or a combination thereof, e.g., a bis(furoylthiourea)benzene derivative, a 2,2′-bith-iophenyl derivative that selectively senses Pb2+ ions. The polymer matrix may be a polyaniline, a polythiophene or the polymer matrix may be an aroylthiourea ionophore inserted into polyvinyl-chloride for Pb2+ and Hg2+ ion sensing.

### TECHNICAL FIELD OF THE INVENTION

The present invention relates in general to the field of ion sensing, and more particularly, to electropolymerizable ligands for selective ion sensing via changes in conductivity.

### BACKGROUND OF THE INVENTION

Without limiting the scope of the invention, its background is described in connection to ion selective electrode sensors. Polymer blends, typically composed of organic ionophores embedded into polymeric materials like polyvinylchloride (PVC), have shown effective sensing of numerous toxic metal ions. However, problems are associated with these types of sensors including poor lower limits of detection (LLOD), decomposition of the polymer component, poor selectivity toward important cations like Pb²⁺, Hg²⁺, and Cd²⁺, and leaching of the plasticizer component.

U.S. Patent Application Publication No. 2014/0047954, entitled, “Method for Thiosulfate Leaching of Precious Metal-Containing Materials,” discloses a process for recovering precious metals from refractory materials using thiosulfate lixiviants. The processes can employ lixiviants that include at most only small amounts of copper and/or ammonia and operate at a relatively low pH, reduction of polythionates, inert atmospheres to control polythionate production, and electrolytic solutions which provide relatively high rates of precious metal recovery.

### SUMMARY OF THE INVENTION

Selective sensing of toxic metal ions, such as Pb(II) ions, in groundwater is most commonly achieved by incorporating selective ligands into the ion selective sensor. The main competition for sensing interaction usually occurs from Hg(II), Cd(II), and Cu(II) ions, which are commonly present with Pb(II) ions. Past work with thiourea derivatives has shown that a number of different mono- and bis-thioureas possess high selectivity towards Pb(II) ions. Aroylthioureas are structurally attractive for interaction with Pb(II) ions due to the soft multi-dentate aprotic binding environment. The synthetic steps to create aroylthioureas are straightforward and versatile, and a library of compounds can be easily produced from a single amine precursor. Selective molecules, such as thioureas, are incorporated into sensors as ionophores in ion selective electrodes (ISEs). Unfortunately, creating long-lasting and accurate sensors can be challenging due to the leaching of plasticizers. The plasticizers serve as a polymeric medium for the ionophore, and leaching into the analyte solution is detrimental to sensor constitution thereby leading to decreased detection limits. The inclusion of thioureas into an electropolymerizable scaffold of the present invention is an effective solution to leaching and stability issues, since the plasticizer will be unnecessary.

Conducting metallopolymers are commonly produced by polymerization of metallated monomers through chemical or electrochemical methods. The effectiveness of post-polymerization synthetic metallation routes have also been studied, with structural analysis techniques. Producing a furanylthiourea electropolymer presents the possibility of the post-polymerization synthetic metallation of the Pb(II) ions. There are many problems associated with metallation after polymerization, commonly from irreversible coordination of commonly used metal catalysts into the polymer. Using electropolymerization as the mechanism of polymerization, especially after a Stille coupling synthetic step, negates the metal poisoning found in other polymers. The inherent conductivity of the metallopolymers allows these materials to be used in a wide variety of applications, from light-emitting materials and drug storage.

The present invention provides an ion selective ligand comprising:

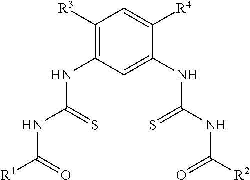

wherein R1 and R2 are independently a furan or a benzene and R3 and R4 are independently a 2,2′-bi(thiophenyl) (BT) or a 3,4-ethylene(dioxy)thiophenyl (EDOT). For example, R1 and R2 be may independently a furan and R3 and R4 are independently a 2,2′-bi(thiophenyl) (BT); R1 and R2 be may independently a benzene and R3 and R4 are independently a 2,2′-bi(thiophenyl) (BT); R1 and R2 be may independently a furan and R3 and R4 are independently a 3,4-ethylene(dioxy)thiophenyl (EDOT); or R1 and R2 be may independently a benzene and R3 and R4 are independently a 3,4-ethylene(dioxy)thiophenyl (EDOT). The ion selective ligand is incorporated into a polymer, polymerized into a polymer or incorporated into an ion selective electrode and may interact with Pb²⁺, Hg²⁺, Cu²⁺, Zn²⁺, Ag⁺, Fe²⁺, Sn²⁺, or Cd²⁺.

The present invention provides a polymeric ion selective ligand comprising one or more aroylthiourea ionophores incorporated in to a polymer matrix to selectively interact with one or more ions. The aroylthiourea ionophores may be poly-5, poly-6, poly-7, poly-7a, poly-7b, poly-8a, poly-8b or a combination thereof and the polymer matrix comprises a polyvinylchloride, a polyaniline, a polythiophene or a combination thereof. The polymer matrix may be an electropolymerizable polymer and may be plasticizer-free. The polymer matrix is ionically porous and allows an electrolyte to intercalate through the polymer. The one or more ions are toxic metal ions may be Pb²⁺, Hg²⁺, Cu²⁺, Zn²⁺, Ag⁺, Fe²⁺, Sn²⁺, or Cd²⁺.

The present invention provides an ion selective electrode comprising an electrode having a coating deposited on the electrode, wherein the coating comprises one or more aroylthiourea ionophores incorporated in to a polymer matrix to selectively interact with one or more ions. The aroylthiourea ionophores may be poly-5, poly-6, poly-7, poly-7a, poly-7b, poly-8a, poly-8b or a combination thereof, e.g., a bis(furoylthiourea)benzene derivative, a 2,2′-bithiophenyl derivative and selectively senses Pb²⁺ ions, a 3,4-ethylendioxythiophenyl derivative and selectively senses Sn²⁺ ions, Pb²⁺ ions, Hg²⁺ ions, Cu²⁺ ions, Zn²⁺ ions, Ag⁺ ions, Fe²⁺ ions, Ce²⁺ ions, or a combination thereof. The polymer matrix may be a polyaniline, a polythiophene or both. For example, the polymer matrix may be an aroylthiourea ionophore inserted into polyvinylchloride for Pb²⁺ and Hg²⁺ ion sensing.

The present invention provides an ion selective electrode (ISE) comprising: a conductive electropolymerizable furanylbis(thiourea) polymer film disposed on a substrate to form an ion selective polymeric sensor to selectively interact with one or more ions. The conducting electropolymerizable furanylbis(thiourea) polymer film may be poly-5, poly-6, poly-7, poly-7a, poly-7b, poly-8a, poly-8b or a combination thereof. The one or more ions are toxic metal ions. The one or more ions are Pb^{2|}, Hg^{2|}, Cu²⁺, Zn²⁺, Ag⁺, Fe²⁺, Sn²⁺, or Cd²⁻. The electrode is a Pt button electrode, an indium tin oxide (ITO) electrode, or a stainless steel electrode.

### BRIEF DESCRIPTION OF THE DRAWINGS

For a more complete understanding of the features and advantages of the present invention, reference is now made to the detailed description of the invention along with the accompanying figures and in which:

FIG. 1 is an image of the synthetic scheme for the monomers 5-8b.

FIGS. 2A-2C are crystal structures of (FIG. 2A) 5 (FIG. 2B) 7b (FIG. 2C) 8b. Hydrogens were omitted from the structure for clarity. FIG. 2D is a graphical representation of the electropolymerized ionophoric film used for selective Pb²⁺ sensors.

FIG. 3A is a Job's plot from ¹H NMR data found for the titration of Pb²⁺ and 7a. FIG. 3B is a Job's plot of 7b with Pb²⁺ indicated a 1:2 binding ratio. FIG. 3C is an example of Benesi-Hildebrand analysis of an UV-Vis titration of 7a with Pb²⁺.

FIG. 4A is a plot of the Electropolymerization of poly-7a from 20 cycles of film growth under oxidative current. FIG. 4B is a plot of the cyclic voltammagrams at scan rates of 10-500 mV/s.

FIG. 5 is an image of a schematic of the M²⁺ electrochemical adsorption/removal process used to test the polymers capacity to interact with the cations.

FIG. 6A is an image of cyclic voltammagrams of poly-5 in the presence of Pb²⁺ ions in 1 M KNO₃ solution. FIG. 6B is a plot of the maximum potential versus the log of each Pb²⁺ concentration (10⁻¹¹ to 10⁻³ M). FIG. 6C is an image of cyclic voltammagrams of poly-7a in the presence of Pb²⁺ ions in 1 M KNO₃ solution. FIG. 6D is a plot of the maximum potential versus the log of each Pb²⁺ concentration (10⁻¹¹ to 10⁻³ M).

FIG. 7 is an image of a cyclic voltammogram of poly-5 (black trace) and the in situ conductivity measurements for poly-5 (red trace) and poly-5 after Pb(II) ion exposure (blue trace).

FIG. 8A is an image of the electrochemical polymerization of poly-5. FIG. 8B is an image of the electrochemical polymerization of poly-7a. FIG. 8C is an image of the electrochemical polymerization of poly-8b.

FIG. 9 is a structure of poly-7a, the polymerization of poly-7a and the interaction of polymerization of poly-7a with Pb²⁺.

FIG. 10 is table of the redox conductivity of the ISE polymer compositions of one embodiment of the invention and an image of the interaction of the polymer and ion.

FIG. 11 is an image of the interaction with poly-8b and Sn²⁺.

FIGS. 12A-12D are images of scan rate dependence studies conducted before and after Pb(II) exposure.

FIG. 13 is a plot showing the distribution of selectivity coefficients for different ions versus log (K_{Pb}^{AMP}).

FIGS. 14A and 14B are plots that denote the redox conductivity (S/cm) for the thiourea polymers before Pb(II) doping and the blue trace shows the conductivity measured for the same film after exposure to Pb(II) ions.

FIG. 15A is a plot comparing the oxidative and reductive scan rate vs. current in the mono(thiourea) poly-8b before and after Pb(II) exposure. FIG. 15B is a plot comparing the oxidative and reductive scan rate vs. current in the bisthiourea poly-5 before and after Pb(II) exposure.

FIG. 16 shows a plot of the absorption under increasing oxidation potential for poly-5.

FIG. 17 is a synthetic scheme to produce compounds 9-13.

FIG. 18A shows the electropolymerization of poly-12 from 20 cycles of film growth under oxidative current. FIG. 18B shows the cyclic voltammagrams at scan rates of 10-500 mV/s in monomer-free solution.

FIG. 19 shows the synthetic route used to produce another electropolymerizable ionophore, 3,8-di(ethylenedioxythien-5-yl) neocuproine. 3,8-bis(ethylenedioxythien-5-yl)-1,10-phenanthroline (EDOT₂phen) was produced via a Stille cross coupling reaction between 3,8-dibromo-1,10-phenanthroline and tri(butyl)stannylethylenedioxy-thiophene with a ⁿBuLi-activated PdCl₂(PPh₃)₂ catalyst.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides novel electropolymerizable furanyl bis (thiourea) compounds that are simple Pb(II) selective polymeric sensors. UV-Vis and NMR titration studies demonstrated that the bis(thiourea) compounds were selective for Pb(II) ions over other competitive metal ions. Cyclic voltammetry studies showed that uniform conducting polymer films, poly-5 and poly-6 could be electrodeposited onto various substrates. Electrochemical studies with the polymers showed selective sequestration of Pb(II) ions out of aqueous solutions by the polymers. XPS studies confirmed that Pb(II) could be extracted from the films, while retaining the film composition. In situ conductivity measurements showed that Pb(II) ions in the polymers increased the conductivity of the films about fifty-fold from 7.75×10⁻² S/cm² to 3.5 S/cm² for poly-5.

The present invention provides a series of thiourea-based monomer and conducting polymer membranes made from a versatile and efficient synthesis. The binding affinities of the furoyl and benzyl(thiourea)s were found by ¹H NMR and spectral titrations. Ancillary groups on the thioureas and the electropolymerizable groups used in the backbone affected the binding affinity significantly in the systems. The monomers with furoylthioureas possessed high selectivity for Pb²⁺ ions, while benzylthioureas were selective towards Cu²⁺ and Sn²⁺ ions as well. Monomers were electropolymerized onto a variety of substrates, and the stability of the films was tested in aqueous conditions. Under oxidative potentials, the polymers adsorbed Pb²⁺ ions and EDTA was then used to remove the ions. The bisthiourea polymers showed no significant degradation or decline in device performance after a year of testing using this washing process. Limits of detection for Pb²⁺ sensing ranged from 9×10⁻⁹-8×10⁻⁸ M for the polymer films. In situ conductivity measurements showed that an enhancement of the electrical conductivity occurred in the polymers in the presence of metal cations.

Polymer blends, typically composed of organic ionophores embedded into polymeric materials like polyvinylchloride (PVC), have shown effective sensing of numerous toxic metal ions. However, problems are associated with these types of sensors including poor lower limits of detection (LLOD), decomposition of the polymer component, and poor selectivity toward important cations like Pb²⁺, Hg²⁺, and Cd²⁺. Plasticizers and resins are the most common media for the ionophores and lead to many of the sensor problems. Leaching of the plasticizer into analyte samples inhibits detection and decreases lifetime of the sensor. Ionophores are tailored to the analyte, with the different binding groups on the ionophore directing the selectivity of the sensor. Modification of the number of binding moieties, the hardness/softness of the moieties, the functional group types, and the steric strain in the ionophore are all variables that affect the selectivity and interaction strength of the ionophore.

Aroylthiourea ionophores inserted into polyvinylchloride served as an effective method for Pb²⁺ and Hg²⁺ ion sensing. Thioketone and amide functionalities combined with the relatively aprotic environment leads to the selectivity of the thioureas. Furthermore, the steric hindrance and donor groups appended to the thioureas allow for tuning of the electronics and local geometry. Another benefit from thioureas is the stabilization provided by intermolecular hydrogen bonding. The hydrogen bonding leads to more rigidity. The hydrogen bonding increases the rigidity in the thiourea leading to more discrete distances for the “binding pocket” in the ionophore. Controlling the size through steric strain and the rigidity can greatly influence selectivity. In other work di(hydroxymethyl) phenanthroline systems, amongst others, were used for Pb²⁺ sensing. Sensors performed more effectively when the ionophores in the sensor systems had binding areas roughly the size of the Pb²⁺ ions in the analyte solutions. To understand the interactions between the ionophore and the metal cation, especially the effect of the binding geometry on selectivity, the speciation must be considered. Bis(thiourea)benzene ionophores, amongst other sensing ligands, are known to interact in a one-to-one mode. Modification of the ancillary groups in this class of ionophores leads to easy manipulation of both sterics and electronics.

Bis(aroylthiourea)benzene ionophores can be easily included into an electropolymer backbone creating a plasticizer-free ISE membrane. Conducting polymers are highly sensitive to the surrounding environment and selective ionophores polymers would benefit not only from this heightened sensitivity, but many of the degradation problems would be negated. LODs for potentiometric Pb^{2|} ISE membrane sensors are near 10⁻⁶ to 10⁻⁷ M. Addition of ionophores into conducting polymers, such as polyaniline and polythiophene enhance the sensitivity of ISEs. Utilizing polymer backbones has led to polymer/plasticizer blends with LODs from 10⁻⁷ to 10⁻⁹ M. The increase in sensitivity is directly linked to the conductivity and electronic properties of the polymers. A polyaniline/ionophore showed remarkable LODs with no decrease in function of the membranes after fifteen months. The sensor utilized not only the added stability, but the ionic permeability of the sensor greatly amplified the capabilities of the ionophore.

The present disclosure also examined the structure, selectivity, and electronic conductivity of electropolymerizable bis(furoylthiourea)benzene in the presence of Pb²⁺ ions. The 2,2′-bithiophenyl derivative selectively sensed Pb²⁺ ions over many other competitive ions. The 3,4-ethylendioxythiophenyl derivative was found to be selective for Sn^{2|} and Pb^{2|} ions. Furthermore, significant increases in the conductivity occurred for each of the electrodeposited polymers when exposed to Pb²⁺ aqueous solutions. Polymer films could be metallated via exposure to Pb²⁺ ions under and oxidative current. XPS of the films indicated that each unit in the polymer was occupied by a Pb²⁺ ion. The metallation proved to be quite facile. Pb²⁺ peaks on the XPS spectra disappeared after the films were exposed to ethylenediaminotetracetic acid (EDTA) solutions. Multiple adsorption/EDTA cycles showed no degradation of the polymer. This initial work indicated that the conducting polymer ionophore films could be effective ISEs.

The diamine reagent used in the synthesis of the electropolymerizable bis(furoylthiourea)benzene compounds can be utilized to create many different bis(aroylthiourea)benzene ionophores. In a similar fashion, monoamine reagents are utilized to produce monothiourea derivatives. Monothiourea ionophores have shown high selectivities for both Pb²⁺ and Hg²⁺ ions, making them very effective ionophores for toxic metal sensing. A total of six thiourea-based, mono- and di-substituted, have been synthesized to study steric and electronic effects on selectivity.

FIG. 1 is an image of the synthetic scheme for the monomers 5-8b. i) fum. HNO₃/H₂SO₄, 0-35° C., 45 min. (74%) ii) a) Pd(PPh₃)₂Cl₂, THF b) 2 equiv. Sn(Bu)₃R, 65° C., 24 hrs. (73-82%) iii) LiAlH₄, THF, Δ, 8 hrs. (45-55%) iv) Fe powder, EtOH, AcOH, THF, Δ, 3 hrs. (57-63%) v) a) NH₄SCN, acetone, benzoyl or furoyl chloride, Δ, 1 h b) acetone, 3, 4a,or 4b, r.t., 2 hrs. (76-95%). Each of the monomers electropolymerized onto a variety of substrates and the sensor response, selectivity, and lifetime of each polymeric ISE was evaluated. Spectroscopic studies of π-π* transitions of the monomers using ultraviolet-visible (UV-Vis) titration experiments were conducted to measure selectivity. Also, through ¹H NMR titration the diamagnetic ions were studied to accurately measure selectivity. Job's plot analysis of the titrations evaluated the speciation of the mono-and di-substituted monomers. Using the Benesi-Hildebrand method, the spectroscopic data can be used to extrapolate binding constants (Kₘ) and binding energies (ΔG_{B}) and probe the strength of the interactions involved in aroylthiourea-M²⁺, which will reveal each monomer's viability as optical and analytical sensors.

TABLE 1
Binding constants and Gibbs free energies of association found through UV-Vis and ¹H NMR titration methods at 294K.

| | 5 | | 6 | | 7a | | 7b | | 8a | | 8b | |
|---|---|---|---|---|---|---|---|---|---|---|---|---|
| | Binding | | Binding | | Binding | | Binding | | Binding | | Binding | |
| | Con- | | Con- | | Con- | | Con- | | Con- | | Con- | |
| | stant | ΔG° | stant | ΔG° | stant | ΔG° | stant | ΔG° | stant | ΔG° | stant | ΔG° |
| Metal | Kₘ | (kJ/ | Kₘ | (kJ/ | Kₘ | (kJ/ | Kₘ | (kJ/ | Kₘ | (kJ/ | Kₘ | (kJ/ |
| Ion | (M⁻¹) | mol⁻¹) | (M⁻¹) | mol⁻¹) | (M⁻¹) | mol⁻¹) | (M⁻¹) | mol⁻¹) | (M⁻¹) | mol⁻¹) | (M⁻¹) | mol⁻¹) |
| ^{a}Ag⁺ | — | — | — | — | — | — | 133 | 11.9 ± 0.1 | 1.6 | 1.2 ± 0.4 | 119 | 11.7 ± 0.2 |
| ^{a}Co⁺² | — | — | — | — | — | — | — | — | 1.7 | 1.3 ± 0.2 | — | — |
| ^{b}Cu⁺² | 147 | 12.2 ± 0.6 | 2.3 | 2.0 ± 0.8 | 820 | 16.4 ± 1.2 | 2224 | 18.8 ± 0.4 | 44 | 14.9 ± 0.2 | 1212 | 17.4 ± 0.5 |
| ^{c}Fe⁺² | 1.7 | 1.3 ± 0.4 | 125 | 11.8 ± 0.3 | 2.8 | 2.5 ± 0.3 | 673 | 15.9 ± 0.7 | 6.6 | 4.6 ± 0.5 | 100 | 11.3 ± 1.3 |
| ^{c}Cd⁺² | 261 | 13.6 ± 0.7 | 4.7 | 3.8 ± 0.2 | 90.0 | 11.0 ± 1.0 | 988 | 16.9 ± 0.4 | 376.9 | 14.5 ± 0.8 | 785 | 16.3 ± 0.3 |
| ^{b}Hg⁺² | 783 | 16.3 ± 0.8 | 115 | 11.6 ± 0.7 | 307 | 14.0 ± 0.4 | 1899 | 18.5 ± 0.2 | 212.6 | 13.1 ± 0.5 | 2376 | 19.0 ± 0.7 |
| ^{b, c}Pb⁺² | 2560 | 19.2 ± 0.2 | 1600 | 18.0 ± 0.9 | 1235 | 17.4 ± 0.4 | 1231 | 17.4 ± 0.8 | 1454.2 | 17.8 ± 0.2 | 2449 | 19.1 ± 1.0 |
| ^{c}Sn⁺² | 1259 | 17.5 ± 0.6 | 1784.3 | 18.3 ± 0.5 | 1111 | 17.1 ± 0.4 | 546 | 15.4 ± 0.2 | 1345 | 17.6 ± 0.4 | 333 | 14.2 ± 0.9 |
| ^{a, c}Zn⁺² | 2.6 | 2.4 ± 0.5 | 55 | 9.8 ± 0.9 | — | — | 234 | 13.3 ± 0.4 | — | — | 137 | 12.0 ± 0.8 |

^{a}Titration experiments had no apparent change/shift in NMR or UV-Vis data.
^{b}Constants and ΔG° values determined by UV-Vis titration methods in ACN.
^{c}Constants and ΔG° values determined by ¹H NMR titration methods in d⁶-ACN.

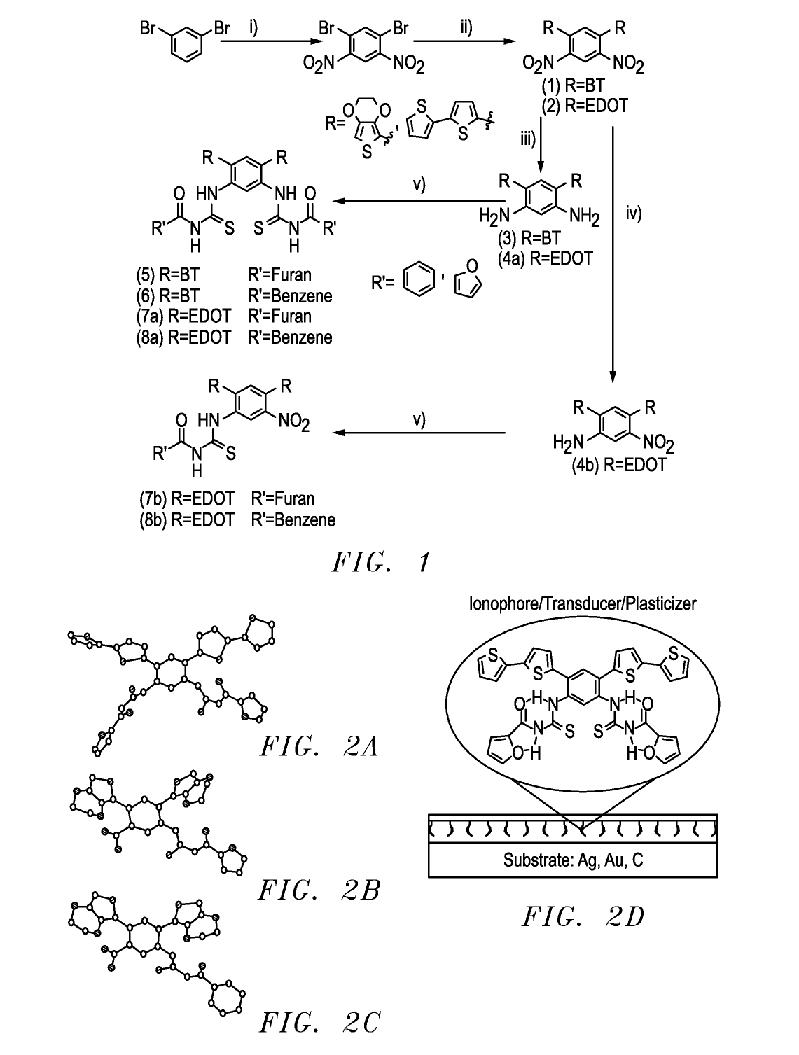

FIGS. 2A-2C are crystal structures of (FIG. 2A) 5 (FIG. 2B) 7b (FIG. 2C) 8b. Hydrogens were omitted from the structure for clarity. FIG. 2D is a graphical representation of the electropolymerized ionophoric film used for selective Pb²⁺ sensors. FIGS. 2A-2C outline the construction of the ion selective sensors made from the thiourea-based polymer films. This ISE would be composed of only the electropolymerized film and the substrate, and earlier work has shown polymer films stable to numerous sensing cycles.

Synthesis and Structure of the Monomers. The synthetic pathways to aroyl and furoyl(bisthioureas) 5-8b started from 4,6-dibromo-1,3-dinitrobenzene which was made in high yield from a simple nitration using fuming nitric acid and sulfuric acid. The precursor material was then used as the halogen partner in a Stille coupling with tri(butyl)stannylbithiophene or tri(butyl)stannyl ethylenedioxythiophene, to form 1, a dark red lustrous semi-crystalline material, or 2, a bright yellow lustrous powder. Both 1 and 2 derivatives were fully characterized by ¹H and ¹³C{¹H} NMR, mass spectrometry, IR spectroscopy and elemental analysis. The nitro derivatives, 1 and 2, were then hydrogenated using activated Fe powder and AcOH/EtOH, producing a green crystalline material, 3, and a pink crystalline material, 4a. ¹H NMR was used to track the formation of NH₂ group, and IR spectra easily showed the disappearance of the nitro groups as a sharp peak at ˜1525 cm⁻¹ to a broad smaller peak at ˜1620 cm⁻¹ and the appearance of a small broad peak at 3535 cm⁻¹.

A selection of bisthioureas could then be synthesized from 3 and 4a, through a high yielding reaction with the desired aroyl thiourea intermediate. The four different bis(thioureas) were purified through recrystallization from DCM and hexanes, and a X-ray quality single crystal was isolated and the crystal structure was found for 5. Due to the large steric bulk around the central benzene, the furoylthiourea and bithienyl groups twist quite significantly out of plane. The amide hydrogen-furoyl oxygen and amide hydrogen-keto oxygen bonding interactions cause the entire furoyl thiourea moieties lie in one plane. Looking at the bond distances for H35-O1 and H37-O3, which are 1.883 Å and 1.926 Å respectively, suggest the furoyl oxygens may form an intramolecular hydrogen bond from the N1 and N3 amide groups. The geometry surrounding the hydrogens is significantly distorted, with 146.8° and 137.2° angles for the N1-H35-O1 and N1-H37-O3 atoms, which further supports intermolecular hydrogen bonding. Similar thiourea structures display this hydrogen bonding forming readily within the thiourea, the extent of this interaction increases with furoyl-type structures. The hydrogen bonding increases the stability of the bis(thiourea)s, with compounds 5, 6, 7a, and 8a increasing chemical and thermal stability. The melting points of the ionophores are near 240° C., and the monomers showed no decomposition in acidic or basic conditions. The crystal structure for 6, showed similar hydrogen bonding between the keto-oxygen and the amide hydrogen. Unlike 5 however, the benzyl is not stabilized and significant twisting occurs in the thiourea portion of the structure.

The hydrogenation of the dinitro compound 2 using LiAlH₄ yielded the aforementioned fully hydrogenated 4a. Changing the reducing agent to Fe powder yielded the partially hydrogenated amine-nitro-compound 4b. Adapting this process to control the partial hydrogenation of 1 proved to be unsuccessful. The formation of 3 was preferred, and the desired precursor to produced BT appended aminonitro precursor could not be produced. The amine precursor 4b was used to produce 7b and 8b using reaction conditions analogous to the bisthiourea synthesis. Crystals were isolated for the monothiourea monomers, 7b and 8b, by slow diffusion of hexanes into chloroform or dichloromethane. The crystal structure for both of the monothioureas showed similar hydrogen bonding found in 5 and 6. Torsion in the backbone and the thiourea portion were found in 8b, with much less order in the unit cell and structure.

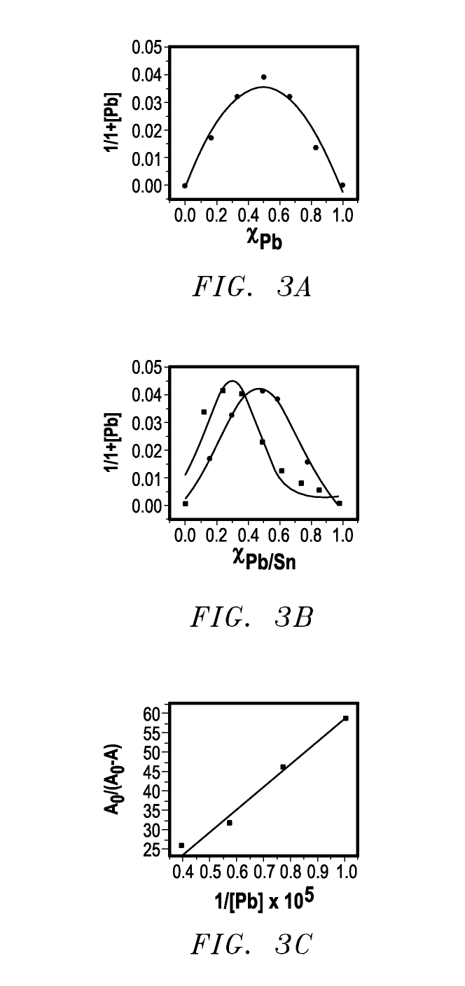

Binding constant determination from various titration methods allows for more information to be gathered on the actual strength and affinity of a metal ion to ionophore system. Numerous studies have used titration experiments monitored by UV-Vis spectrometry and NMR spectroscopy to determine not only the binding constants but the free energies involved in metal ion-system interaction. The appending thiophene-based groups are well known for having strong π-π* transitions in the near ultraviolet-visible range, and 5-8 showed well defined transition bands with high molar absorptivities of 24,000 to 42,000 M⁻¹cm⁻¹. Initial binding experiments with the monomers were conducted to probe the use of UV-Vis spectral methods to study the binding strength of each monomer with any particular metal ion analyte. Introduction of Pb²⁺ ions into a dilute solution of thiourea monomer showed a gradual increase in absorbance as a function of metal ion concentration. Benesi-Hildebrand methods and a Job's plot analysis (FIGS. 3A-3B) of the data demonstrated that there is a host-guest interaction of a 1:1 stochiometry of metal analyte ion to thiourea monomer.

FIG. 3A is a Job's plot from ¹H NMR data found for the titration of Pb²⁺ and 7a. FIG. 3B is a Job's plot of 7b with Pb²⁺ indicated a 1:2 binding ratio. FIG. 3C is an example of Benesi-Hildebrand analysis of an UV-Vis titration of 7a with Pb²⁺. The large steric bulk of the bis(aroylthiourea)s facilitated the 1:1 interaction with the metal ions. Monothiourea-based monomers 7b and 8b mostly interacted in a 1:2 fashion. Based on speciation studies of similar 1-furoylthioureas, this interaction is common for monothioureas with heavy metal cations. FIG. 3 shows a sample Benesi-Hildebrand analysis of a monomer with Pb²⁺ ion used to find the association constant. Binding energies fell into ranges similar to other ligands and ionophores. The monothiourea ionophores were not selective toward Pb²⁺ exclusively like most of the more constrained bis(thiourea) monomers. Monomer 7b was highly selective toward Hg²⁺ and Cu²⁺, and in these cases the interaction occurred in a 1:2 metal to ionophore ratio. 8b was selective toward Pb²⁺ and Hg²⁺, and the Hg²⁺ titrations occurred in a 1:2 ratio.

¹H NMR spectral titrations, monitoring the amide peaks at 9.2-9.5 δ and 12.3-12.5δ, were also used to study the interactions between the monomers and heavy metal cations. The shifts in spectra were analyzed using the Benesi-Hildebrand method and Job's plot analysis. The interactions found for the bisthioureas to the cations in a 1:1 ratio and 1:2 ratio for the monothiourea ionophores. Association constants and energies were in good agreement with those found from the UV-Vis titration data. For instance, ¹H NMR titration showed Pb(II) ΔG°a=19.7 kJ mol⁻¹ for 5 and 18.6 kJ mol⁻¹ for 7a. For the benzylthiourea monomers, ¹H NMR titration showed for Sn(II) binding ΔG°a=20.1 kJ mol⁻¹ for 6 and 16.8 kJ mol⁻¹ for 8a.

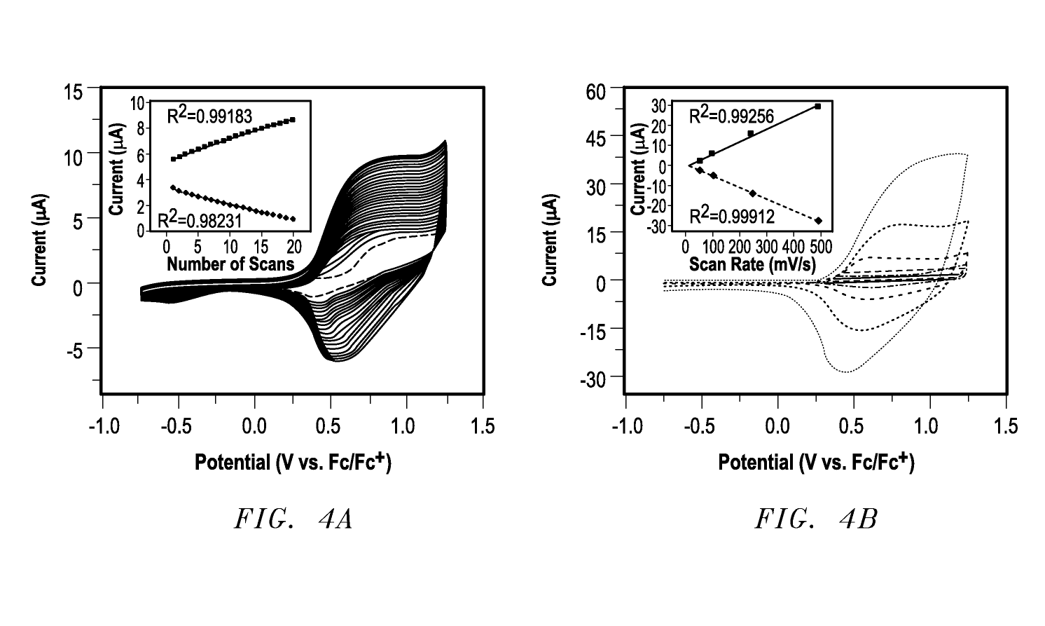

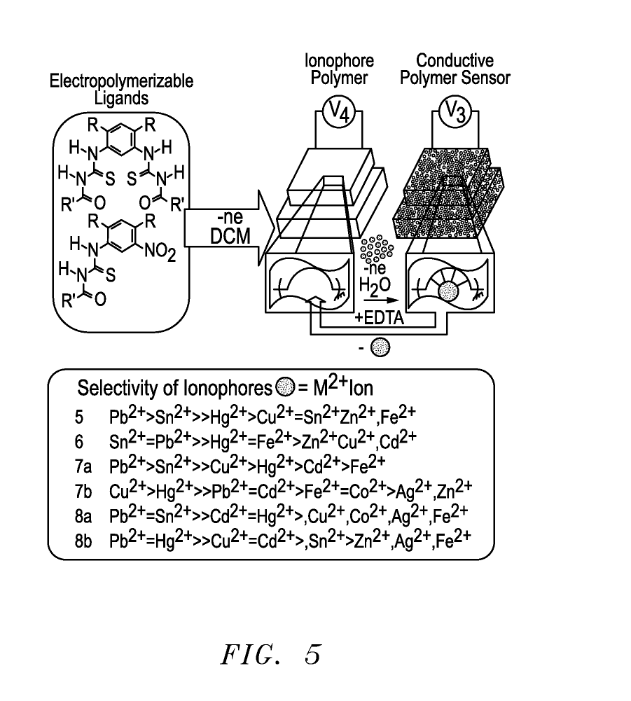

FIG. 4A is a plot of the Electropolymerization of poly-7a from 20 cycles of film growth under oxidative current. FIG. 4A inset is a plot of the film growth dependence showing linear growth of the polymer over the growth cycles. FIG. 4B is a plot of the cyclic voltammagrams at scan rates of 10-500 mV/s. FIG. 4B inset is a plot of linearity of scan rate for oxidative (black trace) and reductive (red trace) scans.

Electropolymerization of Monomers 5-8b. All six monomers were readily electropolymerized under multiple scans in oxidative conditions from −0.5 V to 1.5 V (vs. Fc/Fc⁺) in 0.1 M TBAPF₆ in DCM. FIG. 4 shows electropolymerization data for monomer 7a, representative of all six monomers. Cyclic voltammagrams for the electropolymerization of the monomer can be found in the supporting information. The furoyl type compounds polymerized readily, but an eventual regression in linearity of growth was present in all three cases. All six polymers poly-5-8b, were electropolymerized onto a variety of substrates, Pt button electrodes, stainless steel, and indium tin oxide (ITO) coated glass electrodes. The benzyl class of thiourea polymers was more soluble which may explain more linear polymer growth up to 20 cycles. The scan rate dependence studies showed a linear relationship for all polymers. The linear growth and scan rate dependence reveals that the thiourea polymer films are very electroactive. Also, polymers formed are ionically porous, allowing electrolyte to intercalate through the films promoting further growth. Ionic porosity encourages more homogenous film growth, but also supports metal ions are capable of interacting with the entirety of the film instead of simply the polymer surface. The linear scan rate dependence also indicates that the diffusion of charges throughout the polymer film.

Chemical Stability and Detection of Heavy Metals by the Polymers. Creating a selective ionophore is integral to producing an effective ISE. However, the chemical stability of the sensor is equally important, and many ISEs suffer from low lifetimes. Several experiments were conducted to probe the stability of polymers, poly-5-poly-8b. Initial experiments focused on the performance of the polymers under oxidative aqueous conditions. The polymers on ITO glass substrates were exposed multiple scans between 0 V to 1 V in the presence of Pb²⁺ ions. The films were then washed with an EDTA solution to remove the adsorbed Pb²⁺ ions, completing one cycle. XPS spectra indicated ions adsorbed in an estimated one ion to every polymer unit. This relationship is reflected in the ratio of Pb^{4/5} to S^{2p} peaks for each polymer. Furthermore, based on XPS surface analysis shown in FIG. S7, exposure to EDTA solutions removed all Pb²⁺ ions. All polymers acted similar to the previously reported furanylbis(thiourea)benzene polymers. This process was repeated every two weeks for a year for all polymers, showing no significant decrease in the current. Furthermore, incubation of the polymer films in aqueous solutions showed no detectable degradation of the polymer films on stainless steel.

The polymer ISE sensors were produced by electrodeposition of the polymers as electrode confined films on Pt button electrodes, indium tin oxide (ITO), and stainless steel electrodes. Amperiometric sensing studies were then conducted in conjunction with an Ag/AgCl reference electrode and Pt wire counter electrode. The furanyl-type polymers, poly-5,7a,7b, were tested initially in the presence of a KNO₃ electrolyte. The conducting polymers retained composition under oxidative conditions, and no amperiometric response was observed based on the change in potassium ion concentration. Electrochemical response of the polymer ISEs to Pb(NO₃)₂ was then studied using concentrations from 10⁻²-10⁻¹² M in 1 M KNO₃ solution. A linear increase in current occurred after the lower limit of response (LLOR) was surpassed. Based on the amperiometric response to each analyte concentration, LODs for the polymer were extrapolated.

Stabilization provided by the furanyl group promotes rigidity in the thiourea components of the polymers. The well defined sterics in the ionophores create an optimal site for only Pb(II) or Hg(II) ion interaction. Benzoyl-type polymers, poly-6,8a, and 8b, had different selectivities than the furanyl varieties perhaps due to significant twisting. Conductivity of the polymers films was explored. Similar to past work, each polymer showed significant enhancement in the redox conductivity in the presence of cations.

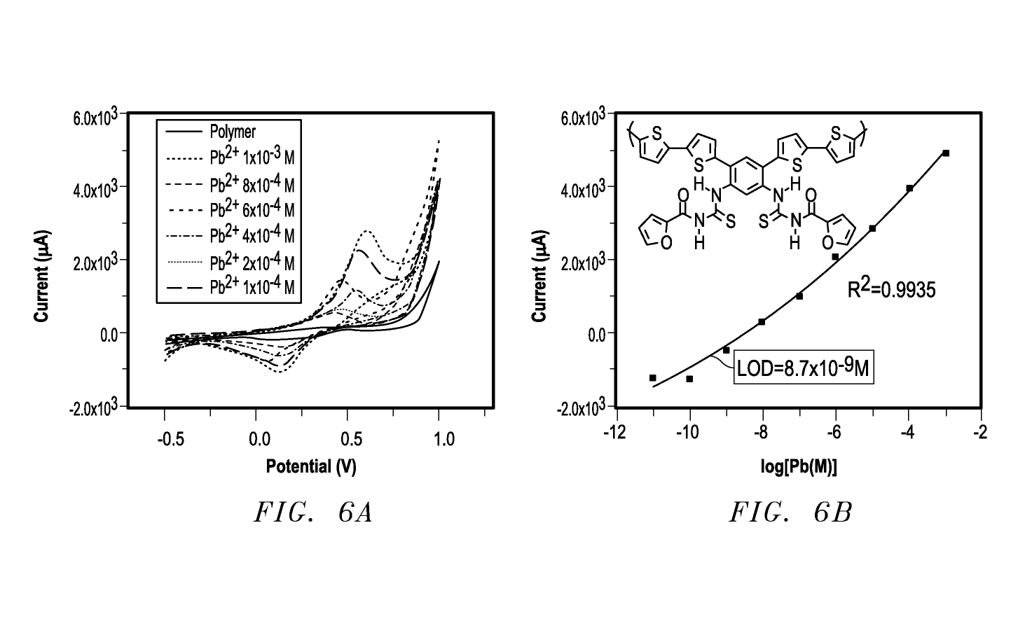

FIG. 5 is an image of a schematic of the M²⁺ electrochemical adsorption/removal process used to test the polymers capacity to interact with the cations.

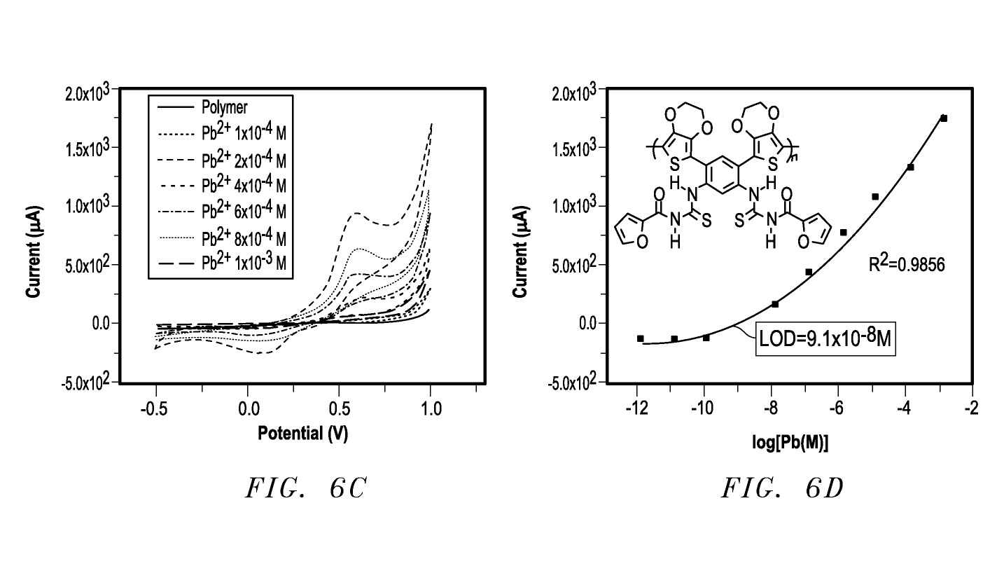

FIG. 6A is an image of cyclic voltammagrams of poly-5 in the presence of Pb²⁺ ions in 1 M KNO₃ solution. FIG. 6B is a plot of the maximum potential versus the log of each Pb²⁺ concentration (10⁻¹¹ to 10⁻³ M). The onset of Nernstian Response was used to find the limit of detection. FIG. 6C is an image of cyclic voltammagrams of poly-7a in the presence of Pb²⁺ ions in 1 M KNO₃ solution. FIG. 6D is a plot of the maximum potential versus the log of each Pb^{2|} concentration (10⁻¹¹ to 10⁻³ M). The onset of Nernstian Response was used to find the limit of detection.

Film thickness was determined using a Veeco Dektak Profilometer. Films were prepared on ITO coated glass that was first treated by successive sonication in deionized water, ethanol, acetone, and methylene chloride. Electropolymerization was done as previously described for 20 cycles at a scan rate of 100 mV/s. Thickness measurements are reported as an average of twelve measurements taken on two films. In situ conductivity measurements were performed by electrodeposition of poly-5 or poly-6 onto an Pt interdigitated electrode purchased from CH Instruments Inc. Films were deposited at scan rate of 100 mV/s for four cycles and following electropolymerization were then studied at a drain offset of 40 mV at a scan rate of 40 mV/s.

Electrochemical syntheses and studies were done under a nitrogen atmosphere in a dry-box using a GPES system from Eco. Chemie. B. V. All of the electrochemical experiments were carried out in a three electrode cell with a Ag/AgNO₃ reference electrode (silver wire dipped in a 0.01 M silver nitrate solution with 0.1 M Bu₄NPF₆ in CH₃CN), a Pt button working electrode (1.6 mm diameter), and a Pt wire coil counter electrode. To calibrate the reference electrode, ferrocene was used as an external reference to which potentials were corrected. All electrochemical experiments were performed CH₂Cl₂ solutions in a supporting electrolyte composed of 0.1 M tetrabutylammonium hexafluorophosphate. The [(n-Bu)4N][PF₆](TBAPF₆) was recrystallized three times from hot ethanol, then dried for 4 days at 100° C. under vacuum.

Crystals of monomer 5 were grown from the slow evaporation of a chloroform solution. The single-crystal diffraction data were collected at 153 K on a on a Rigaku SCX-Mini diffractometer with a Mercury CCD using a Rigaku Tec 50 low-temperature device. The complex was collected using a graphite monochromator with MoKα radiation (λ=0.71075Å). Absorption corrections were applied using Multi-scan. Data reduction was performed using the Rigaku Americas Corporation's Crystal Clear version 1.40.3. The structures were solved by direct methods using SIR974 and refined anisotropically using full-matrix least-squares methods with the SHELX 97 program package. Structure analysis was aided by use of the programs PLATON986 and WinGX7. The coordinates of the non-hydrogen atoms were refined anisotropically. The amine H atoms were located in a difference-Fourier map and both positional and isotropic displacement parameters were refined. All other H atoms were positioned geometrically and refined using a riding model, with C-H=0.95-0.99 Å and with Uᵢₛₒ(H)=1.2 times U_{eq}(C). Neutral atom scattering factors and values used to calculate the linear absorption coefficient are from the International Tables for X-ray Crystallography (1992).

1, 5-Dibromo-2, 4-dinitrobenzene. 1, 3-Dibromobenzene (5.7 g, 24.2 mmol) was added slowly to a stirred mixture of concentrated sulfuric acid (5.3 mL) and fuming nitric acid (5.3 mL) in a 25 mL round bottom flask and cooled in an ice bath. The temperature of the reaction was kept at c.a. 0° C. during addition, and then at room temperature for 40 minutes after the addition was complete. The reaction mixture was then stirred with an excess of crushed ice. A solid yellow precipitate formed and was collected by filtration and washed with water until the filtrate was neutral. The crude solid was recrystallized in a mixture of boiling ethanol (100 mL) and acetone (10 mL) and cooled in the fridge overnight. Two groups of crystals were collected and washed with cold ethanol to give 1, 5-dibromo-2, 4-dinitrobenzene as a bright yellow crystalline solid (5.2 g, 66%) m.p. 117-118° C. (1). To a 200 mL Schlenk flask under inert atmosphere, 1,5-Dibromo-2,4-dinitrobenzene (3.25 g, 10 mmol) and trans-dichlorobis(triphenyl)phosphine palladium (II) (0.35 g, 0.5 mmol) were added. THF (100 mL) was added and the yellow solution was brought to 60° C. and stirred for 15 minutes. 2-(Tributylstannyl)bithiophene 2 (10.0 g, 0.022 mmol) was added slowly to the reaction vessel and the resulting orange solution slowly turned to a dark red, and was stirred under reflux overnight. The solvent was then removed leaving a crude dark solid, which was redissolved in dichloromethane (DCM) and adsorbed onto silica. The crude product on silica was then purified using column chromatography (DCM and hexanes 1:2, 300 mL slurry, R_{f}=0.25). The column separated into three distinct bands with the first being a small amount of quarterthiophene, the second being mono-substituted byproduct, and the third being the desired product, which came off as red solution from which a crystalline red solid readily precipitated (3.65 g, 73.7%) m.p. 176.8° C. (2). To a 200 mL Schlenk flask under inert atmosphere, 1,5-Dibromo-2,4-dinitrobenzene (3.25 g, 10 mmol) and trans-dichlorobis(triphenyl)phosphine palladium (II) (0.35 g, 0.5 mmol) were added. THF (100 mL) was added and the yellow solution was brought to 60° C. and stirred for 15 minutes. 2-(Tributylstannyl)-3,4-ethylenedioxythiophene (9.30 g, 0.022 mmol) was added slowly to the reaction vessel and the resulting light orange solution slowly turned to a dark orange, and was stirred under reflux overnight. The solvent was then removed leaving a crude dark solid, which was redissolved in DCM and dried onto silica. The crude product on silica was then purified using column chromatography (DCM and hexanes 2:1, 6 inch column, R_{f}=0.36). The column separated into three distinct bands with third being the desired product, which came off rose solution that produced a semi-crystalline lustrous yellow precipitate (3.25 g, 72.6%). m.p. 176° C. (3). To a 50 mL three-neck flask under inert atmosphere, 1 (1.00 g, 2.02 mmol) and Fe powder (1.74 g, 24.6 mmol) was added and stirred vigorously for 15 minutes. Then THF (25 mL) was transferred into the flask and the solution was stirred at 50° C. until the red solid dissolved. Acetic acid (8 mL) and dry ethanol (8 mL) was added and the reaction was stirred under reflux for 90 minutes. The solvent was removed from the dark purple solution, leaving a rusty brown precipitate. The crude product was adsorbedonto silica and purified using column chromatography (DCM, 250 mL silica gel, R_{f}=0.15) yielding a green semi-crystalline solid (0.26 g, 29.5%). m.p. 178.3° C. (4a). To a 100 mL three-neck flask in inert atmosphere, 2 (1.00 g, 2.23 mmol) and THF (35 mL) were added. Then LiAlH₄ (840 mg, 22.3 mmol) was added in small portions resulting in a dark red solution. The solution was stirred for two hours resulting in a green solution, and was then stirred overnight at 60° C. The green solution was cooled to room temperature and ethanol was slowly added over 20 minutes, followed by water, to quench any left over LiAlH₄. The resulting orange solution with a grey precipitate was extracted with DCM (3×75 mL) and the organic layer was collected and dried over MgSO₄. The DCM solution was filtered and the solvent was removed in vacuo to yield a dark red precipitate (0.33 g, 38.1%). m.p. 156.4° C. To a 50 mL three-neck flask in N2 atmosphere, 2b (1.00 g, 2.36 mmol) and Fe powder (1.37 g, 24.6 mmol) was added and stirred vigorously for 15 minutes. Then THF (25 mL) was cannula transferred into the flask and the solution was stirred at 50° C. until the yellow solid dissolved. Acetic acid (8 mL) and dry ethanol (8 mL) was added and the reaction was stirred under reflux for 90 minutes. The solvent was removed from the dark red solution, leaving a dark orange precipitate. The crude product was dried onto silica and purified using column chromatography (DCM, 250 mL silica gel, R_{f}=0.40) yielding an orange semi-crystalline solid. (5). To a 50 mL three-neck flask in inert atmosphere, ammonium thiocyanate (0.305 g, 4 mmol) and 10 mL of acetone were added. A solution of furoyl peroxide (0.522 g, 4 mmol) in 5 mL acetone was added dropwise and stirred vigorously for 1 hour under reflux. The foggy white suspension was cooled to room temperature and then 3 (0.436 g, 1 mmol) in 5 mL acetone was added at once. The yellow solution was stirred overnight under reflux, and then cooled to room temperature. After cooling, the solution was added into 50 mL ice water, resulting in a white solution with a yellow precipitate. The aqueous solution was extracted using chloroform (3×50 mL) and the yellow solution was concentrated to 20 mL and placed in a refrigerator. Yellow crystals formed in the flask suitable for single crystal x-ray diffraction after 72 hours (0.60 g, 80.75%). m.p. 236.4° C. (6). Similar procedure to 5, except benzoyl chloride (0.562 g, 4 mmol) was used. Reaction yielded an off-white precipitate (0.70 g, 91.7%). m.p. 237.3° C. (7a). Similar procedure to 5, except the diamine 4a (0.388 g, 1 mmol) was used. Reaction yielded a dark red precipitate (0.554 g, 79.8%). m.p. 210.8° C. (7b). Similar procedure to 5, except the amine 4b (0.418 g, 1 mmol) was used. Reaction yielded an orange precipitate (0.493 g, 85.6%). m.p. 186.9° C. (8a). Similar procedure to 5, except benzoyl chloride (0.562 g, 4 mmol) was used to generate the salt intermediate, and diamine 4a (0.388 g, 1 mmol) was used as the amino reagent. Reaction yielded a dark red precipitate (0.602 g, 84.3%). m.p. 210.9° C. (8b). Similar procedure to 5, except benzoyl chloride (0.562 g, 4 mmol) was used to generate the salt intermediate, and amine 4b (0.418 g, 1 mmol) was used as the amino reagent. Reaction yielded an orange precipitate (0.551 g, 94.7%). m.p. 187.2° C.

One embodiment of the present invention focuses on utilizing a 3,4-ethylene(dioxy)thiophenyl (EDOT) or 2,2′-bi(thiophenyl) (BT) appended furanylbis (thiourea) to create a polymer material, as a solid state sensor. Thiophene-based electropolymerized metallo-polymers are grown linearly due to ionic porosity of the material. Utilizing these porous EDOT and BT-based materials should provide natural pathways for the metal ions to intercalate through and interact with the entire “empty” polymer. A dipole-dipole interaction between the metal ions and the host polymer matrix with a binding energy between 15-40 kJ/mol would be preferred. Sensing could be achieved but irreversible metal coordination would be unlikely. Weak interactions would suggest a lack of both sensitivity and selectivity of the polymer toward the metal ion. A stronger interaction (ΔG≧100 kJ/mol) between the analyte and thiourea polymer would lead to irreversible complexation of the metal and sensor poisoning. UV-Vis and NMR titration studies were performed on the monomers to probe the interaction. Also, determination of the binding energies)(ΔG°) of the metal ions with the monomers can be determined by NMR and UV-Vis spectroscopy. XPS structural analysis was used to test reversibility of the ion uptake process. The effect on the conductivity of Pb(II) ion exposure was measured by studying the furanylbis(thiourea)s using an interdigitated electrode electrochemical study.

The synthesis began with the nitration of 1,3-dibromobenzene using a 1:1 mixture of concentrated sulfuric acid/fuming nitric acid. The yellow crystalline solid was then produced by the stille coupling of 1,3-dibromobenzene to tri(butyl)stannylBT or tri(butyl)stannylEDOT using trans-dichlorobis(triphenyl)phosphino palladium(II) to produce the dinitro-precursor (1), a red solid BT derivative, or (2), a yellow-orange solid EDOT derivative. The diamino-precursor was then synthesized by using Fe powder (3) or LiAlH₄ (4) as a reducing agent. The formation of the diamine (3 or 4) was monitored by the disappearance of the nitro IR bands (1541 (1) or 1524 (2) cm⁻¹) and the formation of an amino IR stretch (3347, 1590 (3) or 3345, 1588 (4) cm⁻¹). The target monomers were synthesized by first creating an excess of the appropriate aroylthiourea salt from furanyl chloride. The diamine precursor was then introduced and after 12 hours under reflux, the target furanylbis(thioureas), 5 or 6, were obtained. A single crystal was isolated, from CHCl₃, of the pale yellow crystalline solid (5) and the X-ray crystal structure was determined. The crystal structure showed in the bithiophene furanyl derivative large deviations in the planarity in both the electropolymerizable bithiophene groups and the thiourea groups. The thiourea portion of the molecule is stabilized by intramolecular hydrogen bonding common in these types of thiourea structures.

The EDOT and BT groups produced strong π-π* transitions that were easily monitored using UV-Vis spectroscopy. UV-Vis titration experiments were performed with a wide variety of metal nitrates in acetonitrile to probe the selectivity of the two monomers. The transition bands increased as the metal ion solutions were added into the host solution. Benesi-Hildebrand analysis of NMR data was used to find the binding constants and Gibbs free energies. Job's plots showed a 1:1 interaction of the guest metal ions with each host monomer. NMR titration studies were performed for all metal ions except the paramagnetic Cu(II) and Hg(II), which were studied using UV-Vis titration analysis. A 0.01 M solution of Pb(NO₃)₂ in d⁶-DMSO was utilized to dissolve both host and guest, and similar ¹H NMR analytical methods were also used, monitoring the δ-δ* of the amide groups at approximately 9.5 ppm and 12.2 ppm. For Pb(II) ions the binding energy was found to be 19.7 kJ.mol⁻¹ for NMR methods. These values agree with the 19.4 kJ mol⁻¹ found in the UV-Vis titration. The monomers are highly selective for Pb(II) ions based on the strength of the interaction of 5 and 6 with typical competitive ions, however 6 showed some competition from Sn(II) ions.

Each monomer was then deposited onto a Pt button working electrode, by cycling from −0.5 V to +1.5 V, to study the aroylbis(thiourea) polymers, poly-5 and poly-6. The conductivity of the polymers was first probed by depositing polymer over 20 sequential scans to test the linearity of polymer growth. A linear relationship was found for current vs. number of scans. The polymer formed is both conductive and ionically porous. Poly-5 and poly-6 both grew uniform films with a linear scan rate and number of scan dependence. Poly-5 and poly-6 were grown onto a stainless steel and indium tin oxide (ITO) coated glass films, and aqueous electrochemical studies were conducted to determine the stability and activity of the polymers in water and in Pb(NO₃)₂ solutions. On both substrates, cyclic voltammetry showed a dramatic increase in current in the presence of Pb(II). Cyclic voltammograms of both polymers display this behavior. The lowest measurable concentration of Pb(II), the limit of detection, for poly-5 and poly-6 was 41 ppb and 20 ppb, respectively. These values are well within 50 ppb, desired by the EPA.

X-ray photoelectron spectroscopy (XPS) was used to study the surface of the polymer films. Control films that were not exposed to Pb(II) ions, films after Pb(II) exposure, and films washed with an EDTA solution were studied. The XPS spectra support that Pb(II) ions were bound into the film, with roughly each furanylbis (thiourea)benzene unit being filled by a Pb(II) ion. When the films were submerged in a solution of EDTA for 5 minutes, over 95% of the Pb(II) was removed from the films. In order to test the stability of the films, polymers were exposed to Pb(NO₃)₂ and washed with EDTA a total of five times, with no decrease in current.

The increase in current seen in the aqueous cyclic voltammograms was further studied by testing the in situ conductivity of the polymers on an interdigitated electrode. There are several ways to test conductivity of polymeric materials, and in situ conductivity methods have been widely used with similar conducting polymers. Electrodes were first calibrated by depositing poly(3-methylthiophene) (p3MT) onto the interdigitated electrodes, and the conductivity was compared to known values. After calibration, the electrode was cleaned and poly-5 and poly-6 were electrodeposited onto the Pt interdigitation in a DCM/TBAPF₆ solution and each terminal was tested. The films were then subjected to cyclic voltammetry in DMF with 0.01M Pb(NO₃)₂ to impregnate the films with Pb(II) ions. After Pb(II) uptake, the treated films were then tested again, looking for an increase in the conductivity similar to the aqueous electrochemical experiments.

FIG. 7 is an image of a cyclic voltammogram of poly-5 (black trace) and the in situ conductivity measurements for poly-5 (red trace) and poly-5 after Pb(II) ion exposure (blue trace). The presence of Pb(II) ions had a significant effect on the conductivity. For poly-5 the conductivity was 7.75×10⁻² S/cm², and for the treated films it was 3.5 S/cm², an almost 50-fold increase in conductivity. For poly-6 the conductivity was 2.48×10⁻² S/cm², and for the treated films 0.7 S/cm². This represents with an almost 30-fold increase in conductivity.

Thienylfuranylbis(thiourea)s of the present invention were synthesized and electropolymerized on various substrates. Electrochemical studies showed that the polymers were both stable in water, and could uptake Pb(II) ions into the polymer. The selectivity and altered electronic conductivity proved that these materials are not only effective Pb(II) sensors, but the Pb(II) ions have a large effect on the conductivity of the polymer.

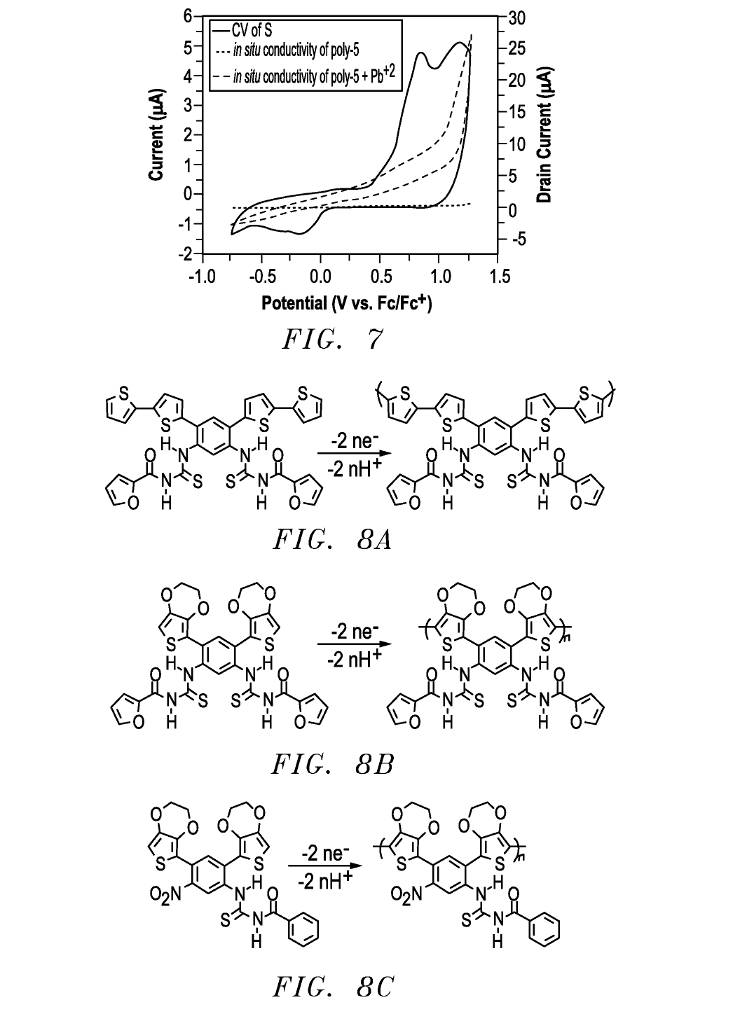

FIG. 8A is an image of the electrochemical polymerization of poly-5. FIG. 8B is an image of the electrochemical polymerization of poly-7a. FIG. 8C is an image of the electrochemical polymerization of poly-8b.

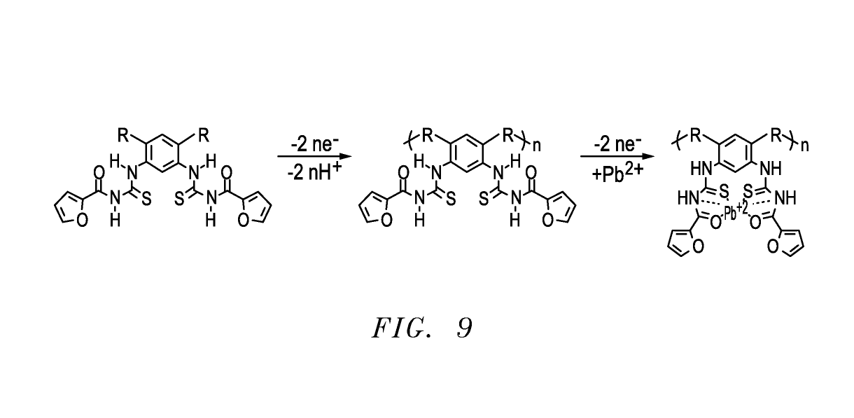

FIG. 9 is a structure of poly-7a, the polymerization of poly-7a and the interaction of polymerization of poly-7a with Pb²⁺.

FIG. 10 is table of the redox conductivity of the ISE polymer compositions of one embodiment of the invention.

FIG. 11 is an image of the interaction with poly-8b and Sn²⁺

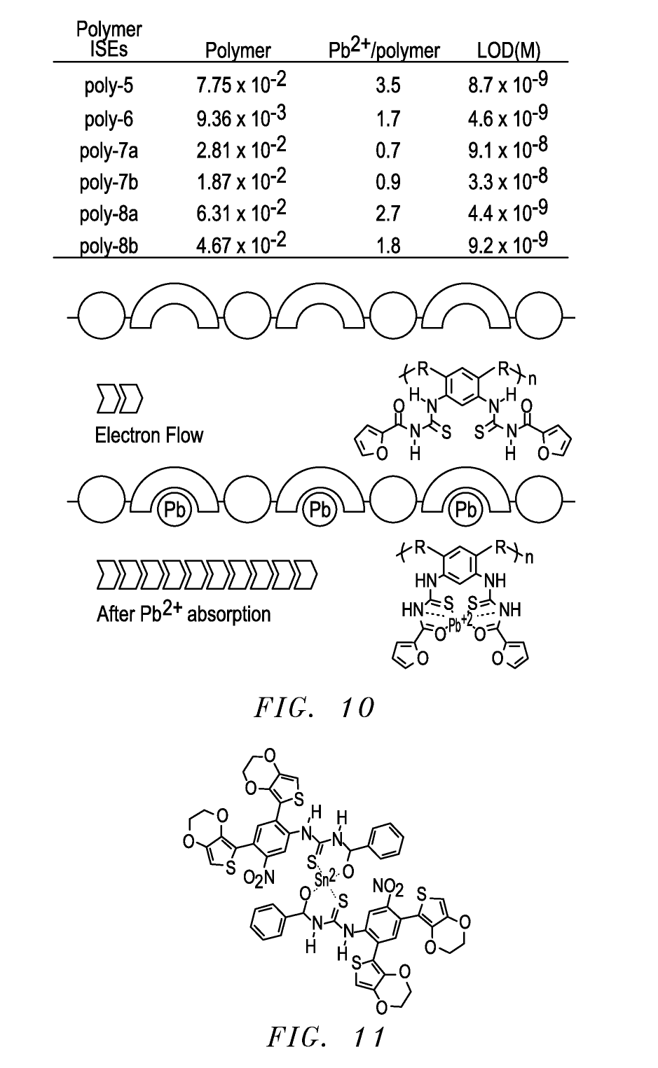

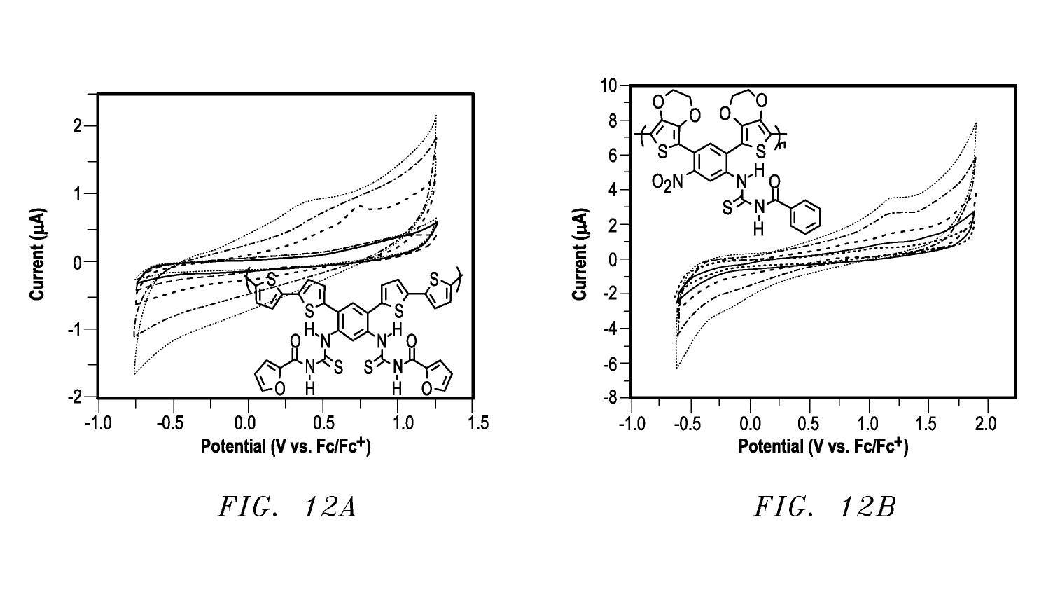

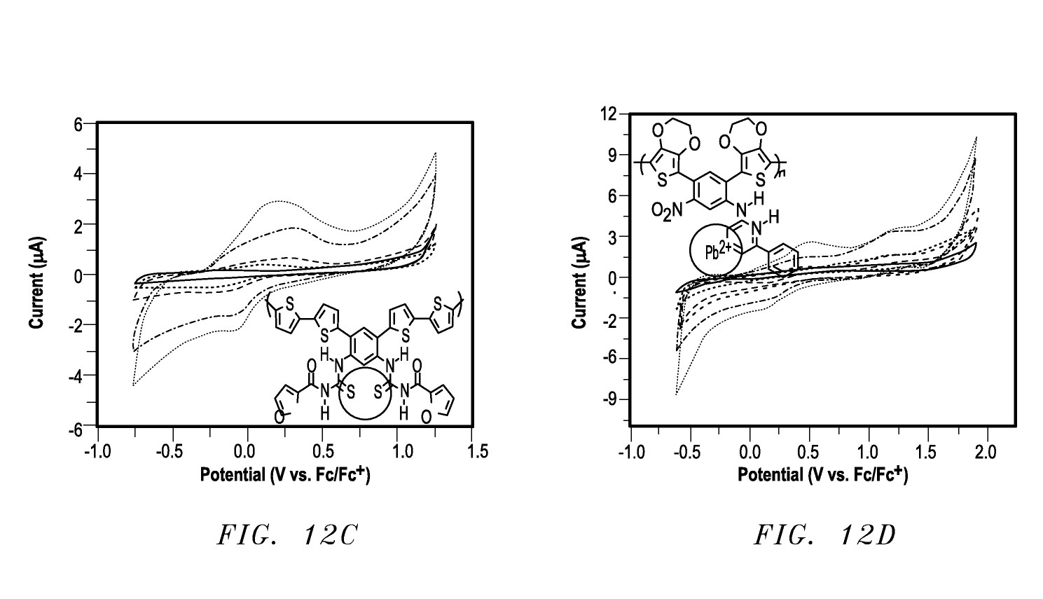

FIGS. 12A-12D are images of scan rate dependence studies conducted before and after Pb²⁺ exposure. The present invention provides an ion selective ligand having the structure:

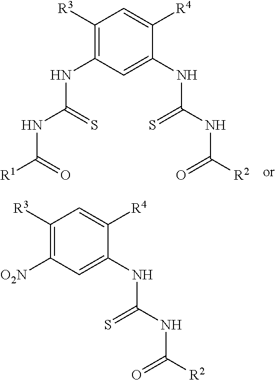

R₁ and R₂ are independently a 5-to 7-membered saturated or unsaturated ring optionally containing one additional heteroatom chosen from N, S, and O, which 5-to 7-membered saturated or unsaturated ring is substituted with 0 to 3 substituents independently chosen from halogen, hydroxy, amino, C₁—C₄ alkyl, C₁—C₄ alkoxy, mono- and di-(C₁—C₄ alkyl)amino, C₁—C₂ haloalkyl, and C₁—C₂ haloalkoxy. The present invention provides an ion selective polymer having the structure:

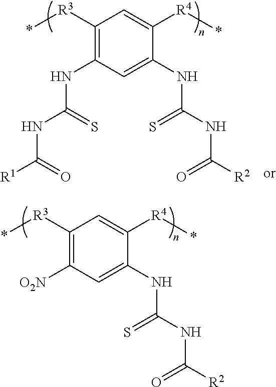

R₁ and R₂ are independently a 5-to 7-membered saturated or unsaturated ring optionally containing one additional heteroatom chosen from N, S, and O, which 5-to 7-membered saturated or unsaturated ring is substituted with 0 to 3 substituents independently chosen from halogen, hydroxy, amino, C₁—C₄alkyl, C₁—C₄alkoxy, mono- and di-(C₁—C₄alkyl)amino, C₁—C₂haloalkyl, and C₁—C₂haloalkoxy.

Monomer selectivity proved to be a good indicator of the selectivity of the ionophoric conducting polymer films. The calibration curves for the polymers were taken by measuring the Ipeak of the CV current under different concentrations of Pb(II) ions. Selectivity coefficients were studied using techniques utilized for ionophore based amperiometric sensors. Coefficients were calculated against currents measured for Pb(II) ions with the polymer electrodes. The following equation adapted from methods of Wang et al. was used to calculate the coefficients for competitive ions:

Iₜ=K(Cᵢ+Σkᵢⱼ^{amp}Cⱼ

where i is the target analyte, It corresponds to the analyte peak current in the presence of the interferents, K is the slope of the calibration curve and Ci and Cj are respectively the concentrations of the analyte and interfering species. Table 2 provides a summary of the coefficients relative to most selective cation found for each ionophore in the monomer studies. Also, FIG. 13 outlines the competitive cations for each polymer, and the level of interference observed for each different ion.

TABLE 2
Conducting Polymer Ionophore

| Ion | 5 | 6 | 7a | 7b | 8a | 8b |
|---|---|---|---|---|---|---|
| Co⁺² | −3.01 | −3.44 | −2.44 | −2.89 | −3.55 | −3.28 |
| Ag⁺ | −2.99 | −3.91 | −3.01 | −3.37 | −4.55 | −4.99 |
| Cu⁺² | −3.45 | −1.03 | −4.03 | −4.44 | −0.56 | −1.44 |
| Cd⁺² | −3.36 | −2.77 | −2.77 | −3.33 | −3.4 | −4.11 |
| Hg⁺² | −1.99 | −2.87 | −2 | −2.78 | 0.23 | −0.99 |
| Pb⁺² | −1.07 | −2.43 | −0.22 | −0.06 | −0.33 | −0.1 |
| Sn⁺² | −3.55 | −3.26 | −3.26 | −3.66 | −3.13 | −3.42 |
| Zn⁺² | −3.01 | −3.44 | −2.44 | −2.89 | −3.55 | −3.28 |

Potentiometric selectivity coefficient K_{pot A, B} = aA/(aB)Z_{A}/Z_{B}. log K_{pot A, B} ≅ 0, strong interference: the sensor responds mainly to the interfering ion; log K_{pot A, B} between −2 and −1, moderate interference; log K_{pot A, B} < −3, no interference.

Stabilization provided by the furanyl group promotes rigidity in the thiourea components of the polymers via hydrogen bonding interaction (vida supra). The well defined sterics in the ionophores created an optimal site for only Pb(II) or Hg(II) ion interaction. Benzoyl-type polymers, poly-6,8a, and 8b, had different selectivities than the furanyl varieties. Based on the selectivity coefficients measured for each polymer, the monomer association energies were a good indicator for polymer selectivity. Strong interference occurred from Cu(II), Sn(II), and Hg(II) ions. FIG. 13 shows relative distribution of selectivity in the polymers, with poly-5 and poly-6 showing good selectivity except versus Sn(II) and Cu(II), respectively. The EDOT-based bis(thiourea) polymers, 7a and 7b showed high selectivity toward Pb(II) ions, with interference occurring mainly from Sn(II) ions. The mono(thiourea) polymers 7b and 8b, however,showed little selectivity toward Pb(II) ions. The heavy interference from Hg(II), Sn(II), and Cu(II) ions demonstrate that the polymers had a higher affinity toward smaller ionic species. Further selectivity measurements versus KampHg showed that the polymers possessed good selectivity toward Hg(II) ions.

The polymer ISE sensors were produced by electrodeposition of the polymers as electrode confined films on Pt button, indium tin oxide (ITO), and stainless steel electrodes. Amperiometric sensing studies were then conducted in conjunction with an Ag/AgCl reference electrode and Pt wire counter electrode. The furanyl-type polymers, poly-5,7a,7b, were tested initially in the presence of a KNO₃ electrolyte. The conducting polymers retained composition under oxidative conditions, and no amperiometric response was observed based on the change in K⁻ concentration. Electrochemical response of the polymer ISEs to Pb(NO₃)₂ was then studied using concentrations from 10⁻²-10⁻¹² M in 1 M KNO₃ solution. Table 3 summarizes the Pb(II) LOD measured for each polymer sensor.

TABLE 3

| | Polymer ISEs | | | | | |
|---|---|---|---|---|---|---|
| | poly - 5 | poly - 6 | poly - 7a | poly - 7b | poly - 8a | poly - 8b |
| LOD | 8.7 × 10⁻⁹ | 4.6 × 10⁻⁹ | 9.1 × 10⁻⁸ | 3.3 × 10⁻⁸ | 4.4 × 10⁻⁹ | 9.2 × 10⁻⁹ |
| (M) | | | | | | |

Initial aqueous electrochemical experiments indicated that the conducting polymer films were sensing Pb(II) ions by a resulting increase in the peak current, Ipeak. Amperiometric behavior in conducting polymers can be linked to several beneficial electronic interactions between the metal and polymer. In the case of the cobalt(I) EDOT-salen type systems, redox matching of the Co I/II redox couple to the oxidative potential of the EDOT polymer backbone led to a dramatic increase in redox conductivity. This led to not only a highly conductive material, but due to the electronic behavior of the Co(I) center with NO and NO₂, an effective amperiometric sensor was estabilished. Similar to this case, Pb(II) doping leads to a dramatic increase in the conductivity of the material. Although this increase is not by virtue of a beneficial redox matching relationship, another mechanism assists in an increase in the redox conductivity.

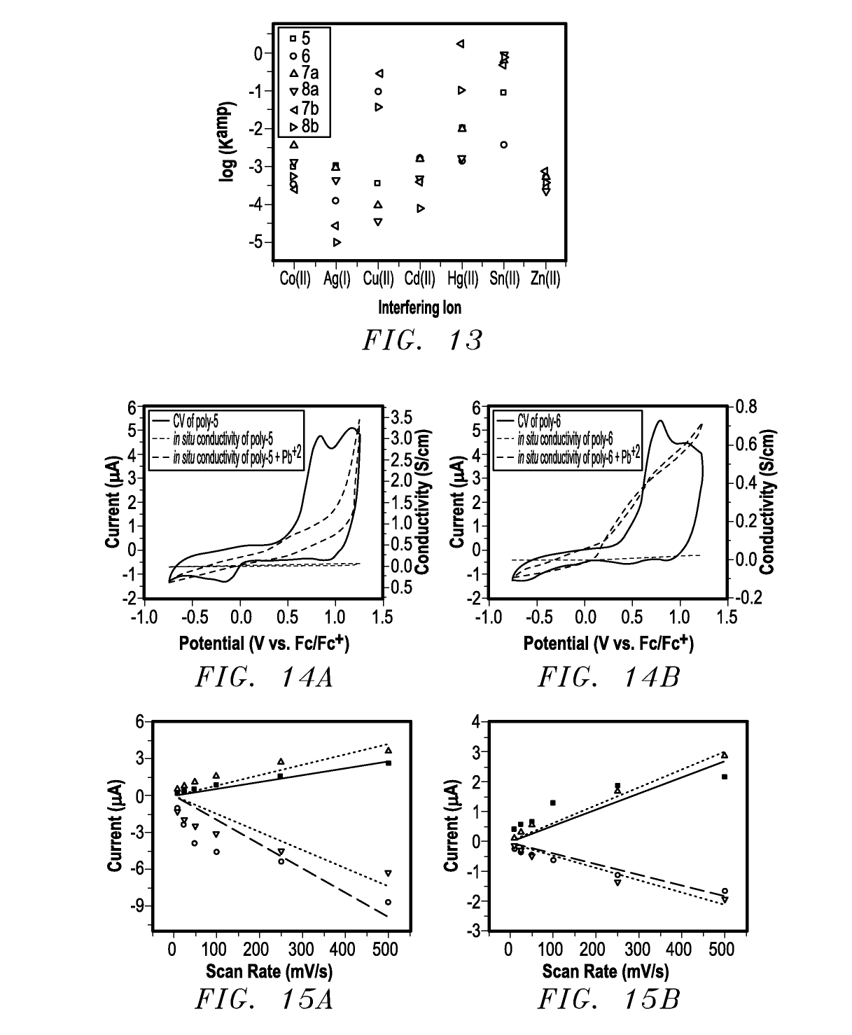

FIGS. 14A and 14B show sample in situ conductivity studies performed on the polymer before and after Pb(II) exposure. Polymers were electrodeposited onto an interdigitated Pt electrode array by scanning from −0.25-1.5 (V vs. Fc/Fc+). The drain current measured was then used to calculate the conductivities using the following equation outlined in literature:

$$σ = \frac{{i}_{D}}{{v}_{D}} \times \frac{D}{n ⋆ T ⋆ L}$$

where iD is the drain current, vD is the offset potential, and T is the polymer thickness, D is the electrode gap (5 μm), n is the number of gaps (149), and L is the electrode length (0.5 cm). Poly-(3-methylthiophene) was used as a calibrating material, and drain currents gathered were readjusted based on the known conductivity of 3-MeTh (σ=60 S/cm). The red trace in FIGS. 14A and 14B denote the redox conductivity (S/cm) for the thiourea polymers before Pb(II) doping and the blue trace shows the conductivity measured for the same film after exposure to Pb(II) ions. FIG. 14 shows (14A) Cyclic voltammogram of poly-5 (black trace) and the in situ conductivity measurements for poly-5 (red trace) and poly-5 after Pb(II) ion exposure (blue trace). (14B) Cyclic voltammogram of poly-6 (black trace) and the in situ conductivity measurements for poly-6 (red trace) and poly-6 after Pb(II) ion exposure (blue trace).

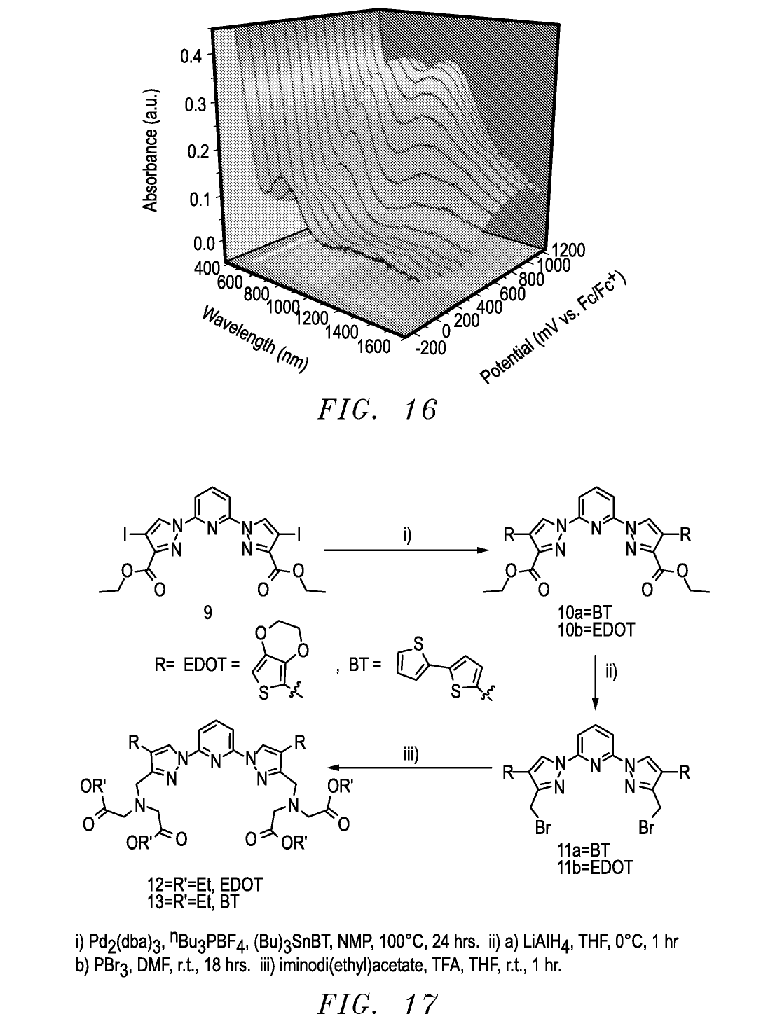

Redox conductivities of the polymers were measured and the ranges of conductivity ranged between 9.36×10⁻³ to 7.75×10⁻² S/cm. Following these measurements each polymer on the interdigitated electrode (IDE) substrate were scanned from −0.5 to +1.5 V in the presence of Pb(II) ions. The in situ redox conductivity measurements were performed again with significant increases in conductivity observed. The conductivity was increased several orders of magnitude in each Pb(II) exposed polymer. Possible explanations of the conductivity increase were studied from two mechanistic pathways. First the increase in conductivity was studied by virtue of the increase in ionic strength. Scan rate dependence studies were performed to study the rate of charge transfer through the polymers. The linearity of the current to the scan rate was then compared to the data gathered for polymers exposed to Pb(II) ions. A sharp increase in the slope of the linear dependence from the polymer to the Pb(II) exposed polymer would indicate the intercalation of Pb(II) ions into the polymer increased conductivity by virtue of the ionic strength. The increase would be related to the Pb(II) ions directly participating in charge diffusion in the polymers. In all cases, there was no significant increase observed in the scan rate dependence studies. FIG. 15A is a plot comparing the oxidative and reductive scan rate vs. current in the mono(thiourea) poly-8b before and after Pb(II) exposure. FIG. 15B is a plot comparing the oxidative and reductive scan rate vs. current in the bisthiourea poly-5 before and after Pb(II) exposure.

Scan rate dependence studies suggested that the ionic strength of the cations had little effect on the conductivity of the polymers. Another possible explanation for the sharp increase in redox conductivity is the Pb(II) metallation changes the planarity of the polymer chains. Crystal structures of the monomers indicated significant torsion and twisting in the aroylthiourea moieties. A similar lack of planarity could exist in the polymers, supported more so by only a small red shift observed in absorption spectra from the monomer (λmax=345 nm) to the polymer (λmax=357 nm) indicating only a slight increase in conjugation as a result of polymerization. Intercalation and binding of Pb(II) ions into the polymers may cause planarization of the polymer structure and therefore increased conjugation favoring formation of the highly conductive quinoidal form upon oxidation.

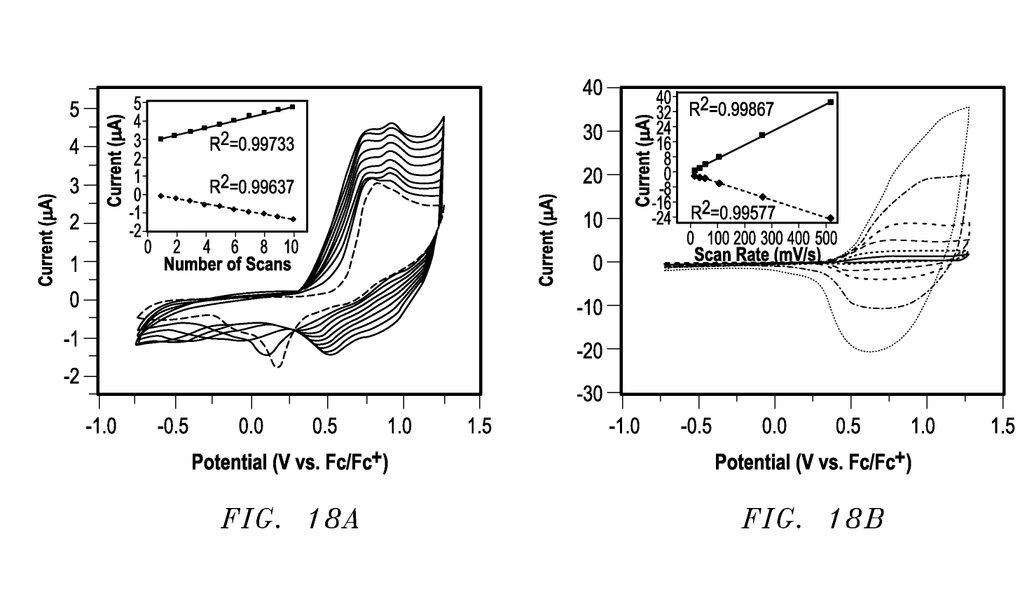

Direct observation of a change in the polymer unit conformation was not possible through surface analysis techniques like TEM or STEM. Instead, spectroelectrochemistry of the polymers was performed to monitor the formation of polaron and bipolaron peaks. FIG. 16 shows a plot of the absorption under increasing oxidation potential. Polaron and bipolaron peaks were observed at ˜850 and 1200 nm, respectively. A distinct shift in the peaks should occur in the presence of Pb(II) if a templating effect is occurring. Spectroelectrochemical experiments were performed after Pb(II) exposure, however, gathering absorption spectra proved to be difficult. The dimethylformamide solvent used in the Pb(II) absorption process delaminated the polymer film from the ITO-coated glass substrate.

Further attempts to understand any changes in conformation induced by Pb(II) ion binding focused on producing Pb(II) complexes from the monomers 5-8b. A variety of Pb(II) salts were used as metal precursors and in each case, the reaction conditions yielded several different products by TLC. Initial purification attempts were attempted by recrystallization. X-ray quality crystals indicated that an alternate process to complexation occurred in the presence of Pb(II) ions.

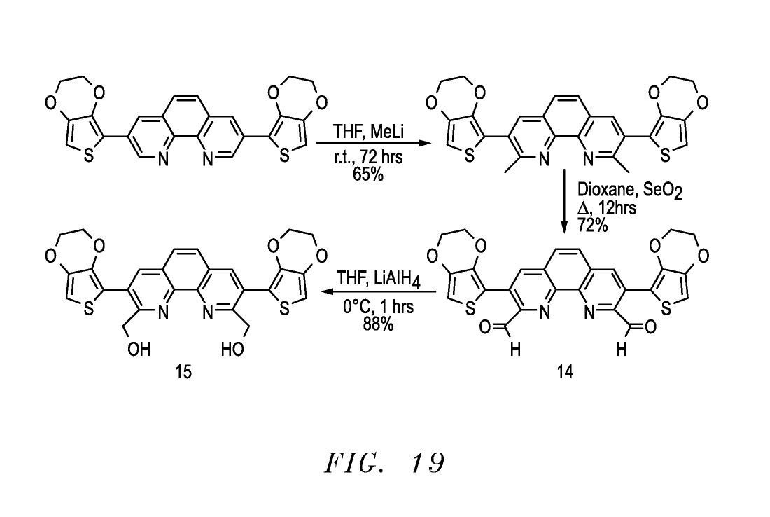

FIG. 17 is a synthetic scheme to produce compounds 9-13. Before the aroylthiourea polymers became a main focus for solid state Pb(II) sensors, another electropolymerizable monomer was synthesized, as shown in FIG. 17. Producing monomers 12 and 13 was synthetically tasking and overall yields in the reaction were approximately 4%. The monomer was effectively polymerized by scanning oxidative potential from −0.25 to +1.25V (FIG. 18).

FIG. 18A shows the electropolymerization of poly-12 from 20 cycles of film growth under oxidative current. (inset) The film growth dependence showing linear growth of the polymer over the growth cycles. FIG. 18B shows the cyclic voltammagrams at scan rates of 10-500 mV/s in monomer-free solution. (inset) linearity of scan rate for oxidative (black trace) and reductive (red trace) scans. Structural differences correlated to selectivity measurements of the monomers, 5-8b, and the conducting polymer ionophores, poly-5-poly-8b, indicated that the local geometry or “binding pocket” of the aroylthiourea moieties governed selectivity. Following this principle, effective polymeric ionophores would be produced from electropolymerizable monomer systems with discrete binding areas. As mentioned earlier, phenanthroline di(methanol) (phen(MeOH)₂) has shown superior affinity toward Pb(II) ions over other ions, such as Zn(II) and Ni(II). This high affinity was explained by geometry of the imine-dimethanol area being roughly equivalent in size to the ionic radius of a Pb(II) ion. Considering this work and some guidelines formed from studying the aroylthiourea systems, a synthetic route was developed to incorporate these ionophores into polymer systems.

FIG. 19 shows the synthetic route used to produce another electropolymerizable ionophore, 3,8-di(ethylenedioxythien-5-yl) neocuproine. 3,8-bis(ethylenedioxythien-5-yl)-1,10-phenanthroline (EDOT2phen) was produced via a Stille cross coupling reaction between 3,8-dibromo-1,10-phenanthroline and tri(butyl)stannylethylenedioxy-thiophene with a ⁿBuLi-activated PdCl₂(PPh₃)₂ catalyst. EDOT₂phen was then saturated in dry THF, and MeLi was added to produce 3,8-bis(ethylenedioxythien-5-yl)-2,9-dimethyl-1,10-phenanthroline. ¹⁷⁸ The methyl groups were oxidized using SeO₂ to render 14, the dialdehyde precursor, and hydrogenation using LiAlH₄ yielded the target molecule 15.

Incorporating ionophoric units into a conducting polymer backbone resulted in highly effective materials for solid state single piece ISEs. The aroylthiourea receptor units were first studied in the electropolymerizable monomers, and it was observed that the ancillary groups on the thiourea moieties and the type of thienyl-based electropolymerizable group changed selectivity. The bulkier EDOT groups directed more preferential sensing of the smaller Sn(II) ion, while the BT groups typically facilitated Pb(II) sensing. The mono(thiourea)-type ionophores demonstrated different selectivities, and may serve as effective Hg(II) ISE materials. Polymer selectivity studies indicated that the conducting polymer ionophores possessed similar selectivites to the parent monomers. Furthermore, these polymers were highly stable under the test conditions and in aqueous environments in the pH range of 2-10. The addition of the conducting polymer to the ionophore greatly increased sensitivity with LODs ranging from 10⁻⁸-10⁻⁸ M. The increased recognition can be directly related to the redox conductivity of these materials in the presence of Pb(II) ions. The redox conductivity in each conducting polymer increased significantly in the presence of the Pb(II) ions from ˜10⁻³-10⁻² S cm⁻¹ to ˜1-5 S cm⁻¹. The LOD for Cd(II) ions was determined to be 4.67×10⁻⁸ M and the LOD for Hg(II) ions was determined to be 1.13 ×10⁻⁹ M using these materials.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

What is claimed is:

1. An ion selective ligand comprising: 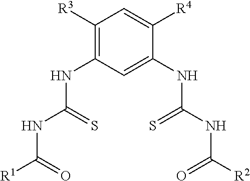
wherein R1 and R2 are independently a furan or a benzene and R3 and R4 are independently a 2,2′-bi(thiophenyl) (BT) or a 3,4-ethylene(dioxy)thiophenyl (EDOT), wherein the ion selective ligand interacts with one or more ions Pb2+, Hg2+, Cu2+, Zn2+, Ag+, Fe2+, Sn2+, or Cd2+.

2. The ion selective ligand of claim 1, wherein R1 and R2 are independently a furan and R3 and R4 are independently a 2,2′-bi(thiophenyl) (BT); R1 and R2 are independently a benzene and R3 and R4 are independently a 2,2′-bi(thiophenyl) (BT); R1 and R2 are independently a furan and R3 and R4 are independently a 3,4-ethylene(dioxy)thiophenyl (EDOT); or R1 and R2 are independently a benzene and R3 and R4 are independently a 3,4-ethylene(dioxy)thiophenyl (EDOT).

3. The ion selective ligand of claim 1, wherein the ion selective ligand comprises poly-5, poly-6, poly-7, poly-7a, poly-7b, poly-8a, poly-8b or a combination thereof.

4. The ion selective ligand of claim 3, wherein the ion selective ligand is incorporated in to a polymer matrix.

5. The ion selective ligand of claim 4, wherein the polymer matrix comprises a polyvinylchloride, a polyaniline, a polythiophene or a combination thereof.

6. The ion selective ligand of claim 4, wherein the polymer matrix is an electropolymerizable polymer or an electropolymer backbone.

7. The ion selective ligand of claim 4, wherein the polymer matrix is plasticizer-free.

8. The ion selective ligand of claim 4, wherein the ion selective ligand is incorporated into an ion selective electrode wherein the polymer matrix is ionically porous and allows an electrolyte to intercalate through the polymer.

9. The ion selective ligand of claim 4, wherein the ion selective ligand is a 2,2′-bithiophenyl derivative and selectively senses Pb2+ ions; or the ion selective ligand is a 3,4-ethylendioxythiophenyl derivative and selectively senses Sn2+ ions, Pb2+ ions, Hg2+ ions, Cu2+ ions, Zn2+ ions, Ag+ ions, Fe2+ ions, Cd2+ ions, or a combination thereof; or the ion selective ligand is a bis(furoylthiourea)benzene derivative.

10. The ion selective ligand of claim 1, wherein the ion selective ligand is inserted into a polymer matrix selected from polyaniline, a polythiophene and polyvinylchloride.

11. The ion selective ligand of claim 10, wherein the polymer matrix is polyvinylchloride for Pb2+ and Hg2+ ion sensing.

12. An ion selective electrode (ISE) comprising:
a conductive electropolymerizable furanylbis(thiourea) polymer film disposed on a substrate to form an ion selective polymeric sensor to selectively interact with one or more ions.

13. The ion selective electrode of claim 12, wherein the conducting electropolymerizable furanylbis(thiourea) polymer film comprises poly-5, poly-6, poly-7, poly-7a, poly-7b, poly-8a, poly-8b or a combination thereof.

14. The ion selective electrode of claim 12, wherein the one or more ions are Pb2+, Hg2+, Cu2+, Zn2+, Ag+, Fe2+, Sn2+, or Cd2+.

15. The ion selective electrode of claim 12, wherein the electrode comprises a Pt button electrode, an indium tin oxide (ITO) electrode, or a stainless steel electrode.
